(19) United States
(12) Patent Application Publication
(10) Pub. No.: US 2005/0019367 A1
(43) Pub. Date: Jan. 27, 2005

(54) Biodegradable implant comprising a polylactide polymer and a lh-rh analogue

(76) Inventors: Jonathan Booth, Cheshire (GB); Rodney Simon Kittlety, Cheshire (GB); Paul Richard Gellert, Cheshire (GB)

Correspondence Address: ROPES & GRAY LLP, ONE INTERNATIONAL PLACE, BOSTON, MA, 02110-2624, US

(21) Appl. No.: 10/489,190

(22) PCT Filed: Sep. 6, 2002
(86) PCT No.: PCT/GB02/04047

(30) Foreign Application Priority Data
Sep. 11, 2001 (GB) 01221134

(51) Int. Cl. A61K 38/09; A61F 2/00

(52) U.S. Cl. 424/426; 514/16

(57) ABSTRACT

A monolithic implant comprising a biodegradable polyester and a luteinizing hormone releasing hormone (LH-RH) analogue. The implant provides continuous release of the LH-RH analogue over a period of six months when the implant is placed in an aqueous physiological-type environment.

### CROSS-REFERENCE TO RELATED APPLICATIONS

This application is a national stage filing under 35 U.S.C. 371 of International Application No. PCT/GB02/04047, filed Sep. 6, 2002, which claims priority from United Kingdom Patent Application No. 0122113.4, filed Sep. 11, 2001, the specification of which is incorporated by reference herein. International Application No. PCT/GB02/04047 was published under PCT Article 21(2) in English

The present invention relates to compositions, more particularly to monolithic implants comprising a biodegradable polyester and a luteinizing hormone releasing hormone analogue (hereinafter LH-RH analogue), which provide continuous release of the LH-RH analogue over a period of at least six months when the implant is placed in an aqueous physiological environment.

LH-RH analogues are used to treat a wide variety of diseases and clinical disorders, for example in the treatment of hormone-dependent tumours such as those found in prostate and breast cancer. However, for efficacy it is necessary maintain a physiologically effective concentration of the LH-RH analogue in vivo over a prolonged period of time.

A number of prolonged release formulations containing LH-RH analogues are known. One approach has been to develop microparticle or microcapsule formulations comprising a biodegradable polymer, typically a poly(lactide-co-glycolide) co-polymer, in which the LH-RH analogue is microencapsulated.

EP 839 525 describes the preparation of microcapsules containing LH-RH analogues using an emulsion based process wherein a water-in-oil emulsion is prepared comprising an inner aqueous phase containing the LH-RH analogue and an outer oil phase comprising a solution of a polymer of lactic acid in an organic solvent. Encapsulation of the inner aqueous phase may then be achieved by one of a variety of methods, including in-water drying of the emulsion, phase separation using a suitable coacervation agent, or by spray drying the water-in-oil emulsion to evaporate the organic solvent from the polymer solution.

Microencapsulation processes such as those described in EP 839 525 require the use of numerous agents and additives in addition to the drug and polymer, for example solvents, surfactants and drug retaining agents etc. These agents and additives can become incorporated into the microcapsules/microparticles, thereby resulting in undesirable contamination of the product. This is especially undesirable when organic solvents, such as methylene chloride, are used in the encapsulation process because many of these are toxic if present in high levels in the microparticles/microcapsules. The removal of such contaminants can be difficult and time consuming requiring, for example, prolonged drying and/or elaborate solvent extraction methods. Particular care is required to ensure that the extraction/drying processes employed do not result in degradation of the microparticles/microcapsules through, for example coagulation of the particles or a degradation of the release profile of the drug from the microparticle/microcapsule. Furthermore, microencapsulation processes can be very sensitive to changes in process parameters which makes them difficult to scale up for large scale production of microparticles.

Generally, microencapsulated LH-RH analogues are suitable for delivering the LH-RH analogue for up to 3 months. To provide treatment over a longer duration it would be necessary to give patients a large dose of the LH-RH analogue and therefore administer a large number of microparticles/microcapsules. However, microparticles/microcapsules are administered to patients as a suspension in a liquid medium, therefore, the large number of microparticles required to achieve a longer treatment duration would necessitate the injection of a large volume of material to a patient. Many patients would find such a treatment regimen painful and unacceptable compared to the conventional monthly or three monthly treatments. An alternative approach would to use a high loading of the LH-RH analogue in the microparticle/microcapsule to ensure that a therapeutically effective dose of the LH-RH analogue is maintained whilst minimising the volume of material required for injection. However, if the loading of the LH-RH analogue is too high this can result in a poor, uncontrolled, release profile of the LH-RH analogue.

EP 058 481 describes monolithic implants comprising a biodegradable poly(lactide-co-glycolide) co-polymer and an LH-RH agonist. EP 058 481 describes the release of an active ingredient contained in an implant comprising a poly(lactide-co-glycolide) copolymer occurring in two distinct phases. Firstly a diffusion phase in which release occurs essentially by diffusion of the active out of the polymer matrix followed by a degradation phase in which the biodegradable poly(lactide-co-glycolide) copolymer is broken down by hydrolysis. Continuous release of the active agent is achieved by selecting a poly(lactide-co-glycolide) copolymer whereby the diffusion and degradation phases overlap, thereby avoiding “flat spots” in the release profile in which little or no drug is released.

The implants described in EP 058 481 can be implanted directly into patients without the need for a suspension medium, thus reducing the volume of material injected compared to microparticle/microcapsule delivery systems.

However, the maximum duration of release of the implants described in EP 058 481 is three months. Therefore, it is necessary for patients to have new implants fitted on a regular basis. Frequent replacement of implants requires intervention by medical practitioners. There is, therefore, a need for a monolithic implant which provides continuous release over a longer period of time, thereby reducing the frequency of re-implantation.

We have surprisingly found that monolithic implants prepared using a combination of a specific polylactide polymer and a LH-RH analogue loading provides a monolithic implant which continuously releases the LH-RH analogue over a period of at least six months when placed in an aqueous physiological-type environment.

According to a first aspect of the present invention there is provided a monolithic implant comprising:
(i) a polylactide polymer having a weight average molecular weight of from 12,000 Daltons to 40,000 Daltons; and
(ii) from 25 to 40% by weight based upon the total weight of the implant of LH-RH analogue;
   wherein the monolithic implant continuously releases the LH-RH analogue over a period of at least six months when placed in an aqueous physiological-type environment.
   Polylactide Polymer

The polylactide polymer is a homopolymer wherein all the repeat units of the polymer are of the Formula (1): 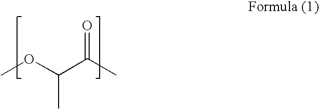
The repeat units may be in the L-, D- or a mixture of the L- and D-configurations. Preferably the repeat units of Formula (1) comprise a mixture of L- and D-configurations. When the polymer comprises a mixture of repeat units in the L- and D-configurations the ratio of L- to D-units in the polymer is preferably from 25:75 to 75:25, more preferably from 30:70 to 70:30 and especially approximately 1:1.

Each polymer chain is preferably terminated by one hydroxy group and one —COOH group. However, in embodiments of the present invention other terminal groups may be present, provided that the presence of such terminal groups do not adversely affect the release of the LH-RH analogue from the implant. Suitable terminal groups other than —OH or —COOH which may be present on the polymer include esters formed by reacting an appropriate acid or alcohol with the —OH and/or —COOH end group(s) of the polymer. Suitable esters include alkyl (preferably C₁₋₄-alkyl) or aralkyl (preferably benzyl) esters.

Preferably the polymer carries at least one —COOH group because this provides a more favourable release profile, particularly when the LH-RH analogue contains one or more basic groups such as an amino group.

Additional —COOH groups may be incorporated into the polymer to modify the release profile of the LH-RH analogue from the implant, especially when the LH-RH analogue contains one or more basic groups. Additional —COOH groups may be introduced by, for example, reaction of a suitable polycarboxylic compound or anhydride thereof with the terminal hydroxy and/or —COOH group(s) of the polymer.

Polycarboxylic compounds suitable for reaction with a terminal hydroxy group of the polymer include polycarboxylic acids and anhydrides thereof, for example malonic acid, succinic acid, glutaric acid or adipic acid; or hydroxypolycarboxylic acids and anhydrides thereof, for example malic acid, citric acid, glutaric acid or glutaric anhydride.

Polycarboxylic compounds suitable for reaction with a terminal —COOH in the polymer include the hydroxypolycarboxylic acids hereinbefore mentioned.

Suitable methods for incorporating additional —COOH into a biodegradable polyester are described in U.S. Pat. No. 5,863,985 which is incorporated herein by reference thereto.

The weight average molecular weight of the polylactide polymer is preferably from 12,000 to 35,000 Daltons, more preferably from 18,000 to 33,000 Daltons, especially from 21,000 to 31,000 Daltons. The weight average molecular weight (MW) of the polymer is measured using size exclusion chromatography (SEC) using polymer solutions in Tetrahydrofuran (THF) with 2×30 cm² mixed bed ‘D’s PLGel columns (supplier Polymer Laboratories) which have a linear range of MW 200-400,000 Da; wherein the system is first calibrated using PL Easical PS-2 polystyrene calibrants with MW's in the range 580 to 400,000 Da. The PLGel packing material is a highly cross linked spherical polystyrene/divinylbenzene matrix. A Wyatt Optilab DSP refractometer may be used for detection.

The polylactide polymer may comprise a single polylactide homo polymer or a blend of two or more polylactide homo polymers. A blend of two or more polylactide polymers can be used to provide further control over the rate of release of the LH-RH analogue from the implant, thereby providing a more consistent rate of release over the life-time of the implant in a physiological type environment. Blends of polymers are particularly useful for minimising “flat spots” in the LH-RH analogue release profile, thereby providing a smooth, steady release of the LH-RH analogue from the implant.

When the polymer comprises a blend of two or more polylactide polymers the individual polylactide polymers components of the blend are selected to provide a mean weight-average molecular weight of the blend in the range of from 12,000 Daltons to 40,000 Daltons. By way of example, the blend may comprise 75 parts by weight of a 21,000 Daltons polylactide and 25 parts by weight of a 31,000 Daltons polylactide polymers, thereby giving a mean weight-average molecular weight of the blend as 23,500 Daltons. Similarly a 1:1:1 blend of polylactide polymers with molecular weights of 12,500, 31,000 and 59,000 provides a mean weight-average molecular weight of 34,170 Daltons.

When the polymer comprises a blend of polylactide polymers it is preferred that the maximum molecular weight of any one component of the polymer blend does not exceed 60,000 Daltons. More preferably, each component of the polymer blend has a weight-average molecular weight in the range of from 15,000 Daltons to 40,000 Daltons because we have found that this molecular weight range provides a steady and continuous rate of release of the LH-RH analogue from the implant.

The polylactide polymer may be prepared using known methods. Suitable techniques include but are not limited to condensation polymerisation of lactic acid and ring-opening polymerisation of lactide (3,6-dimethyl-1,4-dioxane-2,5-dione). The lactic acid or lactide used in the polymerisation process may be the L-, D- or more preferably a mixture of the L- and D-isomers and especially a racemic mixture.

Condensation polymerisation of lactic acid may be performed using a variety of methods well known to a person of ordinary skill in the art. For example, lactic acid may be heated under reduced pressure optionally in the presence of a suitable catalyst whilst removing water by distillation. The condensation reaction may be performed at a temperature of from 100 to 180° C. The pressure during the condensation reaction is preferably reduced to from 30 mm Hg to 1 mm Hg. Suitable reaction times are typically from 2 to 10 hours. Suitable catalysts when used include solid inorganic acids such as an acid clay, an acid ion-exchange resin or stannous octanoate. Suitable condensation polymerisation reactions are described in EP 172 636 and Synthesis of Polylactides, Polyglycolides and Their Copolymers, Nieuwenhuis, Clinical Materials 10, 1992 59-67.

A preferred method for the preparation of the polylactide polymer is ring-opening polymerisation of lactide. The ring opening polymerisation is performed under conditions of elevated temperature and in the presence of a suitable catalyst using conditions well known in the polymer art.

Suitable catalysts for the ring-opening polymerisation include but are not limited to zinc, zinc oxide, zinc chloride, p-toluene sulphonic acid, antimony catalysts, for example antinomy trifluoride, or organo-tin catalysts, for example stannous octoate (stannous 2-ethylhaxanoate) or tin chloride.

A suitable reaction temperature for the ring-opening polymerisation is from about 120° C. to about 240° C., more preferably from 140° C. to 200° C. The ring opening polymerisation is preferably performed over a period of from 1 to 10 hours more preferably from 2 to 6 hours.

Preferably the ring opening polymerisation reaction is performed in the presence of a suitable chain termination agent thereby controlling the MW of the resultant polylactide polymer. Suitable chain termination agents include water, a hydroxy-carboxylic acid such as lactic acid or an alcohol, such as a C₁₋₆-alkanol. It is especially preferred that the chain termination agent is lactic acid or a mixture of lactic acid and water.

The methods used to prepare polylactide polymers typically results in a mixture of individual polylactide polymer chains, many of which are of differing chain lengths. The polydispersity of a polymer provides an indication of the spread of chain lengths in such a mixture and is defined to be the ratio of the weight average molecular weight (MW) to the number average molecular weight (Mₙ). Suitably, the polydispersity of the polylactide polymer is from 1.3 to 4.5.

LH-RH Analogue

Preferably the monolithic depot according to the present invention comprises from 25 to 35%, more preferably from 28 to 33% and especially approximately 30% by weight of the LH-RH analogue.

The LH-RH analogue may be an LH-RH agonist or a pharmaceutically acceptable salt thereof, or an LH-RH antagonist or a pharmaceutically acceptable salt thereof. Preferred LH-RH analogues are peptides or peptide derivatives.

Examples of suitable LHRH agonists include, but are not limited to;
i) buserelin (U.S. Pat. No. 4,024,248)
   (pyr)Glu-His-Trp-Ser-Tyr-D-Ser(Bu^{t})⁶-Leu-Arg-Pro-NHCH₂CH₃
ii) triptorelin (U.S. Pat. No. 4,010,125)
   (pyr)Glu-His-Trp-Ser-Tyr-Trp-Leu-Arg-Pro-Gly-NH₂
iii) leuprorelin (U.S. Pat. No. 4,005,063)
   (pyr)Glu-His-Trp-Ser-Tyr-D-Leu-Leu-Arg-Pro-NHCH₂CH₃
iv) goserelin (U.S. Pat. No. 4,100,274)
   (pyr)Glu-His-Trp-Ser-Tyr-D-Ser(Bu^{t})⁶-Leu-Arg-Pro-(Azygly)NH₂
v) deslorelin (U.S. Pat. No. 4,659,695)
   (pyr)Glu-His-Trp-Ser-Tyr-D-Trp-Leu-Arg-Pro-NH—CH₂—CH₂—NH₂
vi) histerelin (U.S. Pat. No. 4,244,946)
   (pyr)Glu-His-Trp-Ser-Tyr-D-His(Bzl)-Leu-Arg-Pro-NH—CH₂—CH₃
vii) avorelin (U.S. Pat. No. 5,668,254)
   (pyr)Glu-His-Trp-Ser-Tyr-D-Trp(2-Me)-Leu-Arg-Pro-NH—CH₂—CH₃
viii) nafarelin (U.S. Pat. No. 4,234,571)
   (pyr)Glu-His-Trp-Ser-Tyr-D-Nal(2)-Leu-Arg-Pro-NH—CH₂—CH₃;
      lutrelin, cystorelin, gonadorelin or detirelix.

Preferably the LH-RH agonist is selected from leuprorelin, buserelin, triptorelin and goserelin. It is especially preferred that the LHRH agonist is goserelin.

Examples of suitable LHRH antagonists include, but are not limited to, antide, antarelix, cetrorelix, azaline, ganirelix and those disclosed in U.S. Pat. No. 5,470,947 (Folkers); U.S. Pat. Nos. 5,413,990 and 5,300,492 (Haviv); U.S. Pat. No. 5,371,070 (Koerber); U.S. Pat. No. 5,296,468 (Hoeger); U.S. Pat. No. 5,171,635 (Janaky); U.S. Pat. Nos. 5,003,011 and 4,431,635 (Coy); U.S. Pat. No. 4,992,421 (De); U.S. Pat. No. 4,801,577 (Nestor); and U.S. Pat. Nos. 4,851,385, 4,689,396 and 5,843,901 (Roeske).

When the LH-RH agonist or antagonist is in the form of a salt the salt may be one formed with a suitable acid or basic group in the LH-RH analogue.

Suitable salts which may be formed with basic group(s) in the LH-RH analogue include, for example salts formed with inorganic acids (for example, hydrochloric acid, sulphuric acid or nitric acid), organic acids (for example, carbonic acid, bicarbonic acid, succinic acid, acetic acid, propionic acid or trifluoroacetic acid) etc. Preferably, the salt is a salt formed with an organic acid (for example carbonic acid, bicarbonic acid, succinic acid, acetic acid, propionic acid, trifluoroacetic acid), especially a salt formed with acetic acid.

Suitable salts which may be formed with acidic group(s) in the LH-RH analogue (such as carboxy groups) include salts formed with an alkali metal (such as sodium and potassium), alkaline earth metal (such as magnesium and calcium), aluminium and ammonium salts, as well as salts with suitable organic bases such as ethanolamine, methylamine, diethylamine, isopropylamine, trimethylamine and the like.

It is especially preferred that the LH-RH analogue is an LH-RH agonist or a pharmaceutically acceptable salt thereof, more preferably goserelin or a pharmaceutically acceptable salt thereof, especially the acetate salt.

Monolithic Implant

The monolithic implant according to the present invention comprises a unitary structure, typically having dimensions greater than about 0.5 mm, more preferably from 1 mm to 30 mm. The geometry of the implant will depend upon the method used to prepare the implant, the means used to insert the implant in a patient and the dose of LH-RH analogue required. Suitable geometries include slabs, cylinders, rods, spheres or pellets. In a preferred embodiment the implant comprises a cylindrical rod because this provides a convenient geometry for sub-dermal implantation into a patient using a needle or conventional surgical instruments such as a trochar.

The monolithic implants according to the present invention continuously release the LH-RH analogue over a period of at least six months when placed in an aqueous physiological-type environment.

The term “continuous” as used herein refers to a continual release of LH-RH analogue from the implant for at least six months after implantation into an aqueous physiological environment. The rate of release of the LH-RH analogue may vary during the six month period, for example a short “initial burst” of LH-RH may be observed shortly after implantation of the implant. However, there are no periods in the six months following implantation in which there is little or no release of LH-RH analogue from the implant. Preferably at least 0.05% by weight, more preferably greater than 0.1% by weight of the LH-RH analogue is released per day when the implant is placed in an aqueous physiological environment. Preferably, the rate of release of LH-RH analogue is approximately constant, but always continuous, over most of the six month release period.

The term “aqueous physiological environment” as used herein refers to the body of a warm blooded animal, particularly man, and especially the subcutaneous environment of such a body. These conditions may be simulated in vitro by placing an implant in an aqueous dissolution medium, optionally buffered to a physiological pH, at a temperature of from 35 to 40° C. A suitable dissolution medium comprises a saline solution buffered to a pH of approximately 7.4 using a phosphate buffer, for example phosphate buffered saline or Mcllvaines citric acid phosphate. Preferably, the aqueous dissolution medium is maintained at a temperature of 37° C. ±2° C. The amount of LH-RH analogue released over a given time period may be determined by sampling the dissolution medium and measuring the concentration of LH-RH analogue using a suitable analytical method, for example HPLC.

According to a further aspect of the present invention there is provided a medicament comprising a first monolithic implant according to the present invention and a second monolithic implant according to the present invention, wherein the first and second implants release the LH-RH analogue at different rates when placed in an aqueous physiological environment.

The use of a combination of two or more different implants according to the present invention enables a wide range of release profiles to be achieved by appropriate selection of polymers and/or loading of the LH-RH analogue. This may be advantageous for the treatment of certain diseases. For example, it may desirable to provide a high initial dose of LH-RH, followed by a lower dose for the remainder of the treatment. This may be achieved by selecting a first implant which has a high initial release rate of LH-RH analogue and a second implant which has a more constant release rate. The cumulative LH-RH analogue release from the two implants thereby providing a high initial dose followed by a substantially constant release rate for the remainder of the treatment period. Alternatively, by appropriate selection of two or more different implants it is possible to provide a cumulative release of LH-RH analogue which is substantially zero order (i.e. substantially constant) throughout the treatment period.

The release profile of LH-RH analogue from the first and second implants may be controlled by, for example, varying the molecular weight of the polylactide and/or the loading of the LH-RH analogue in the implant.

The first and second implants may be implanted (preferably subcutaneously) at the same or different parts of a patients body.

According to a further aspect of the present invention there is provided a process for the preparation of an implant according to the present invention comprising the steps:
(i) preparing a solution or dispersion of the LH-RH analogue in a solution comprising the polylactide polymer and a suitable organic solvent;
(ii) removing substantially all of the organic solvent from the solution or dispersion formed in step (i); and
(iii) forming the product of step (ii) into a monolithic implant of the required dimensions.

Preferably the LH-RH is dissolved in the polymer-organic solvent solution in step (i) of the process. A suitable organic solvent for use in step (i) includes, for example glacial acetic acid.

The organic solvent may be removed in step (ii) of the process using any convenient technique, for example by evaporation or freeze-drying. Preferably at least 80% by weight, more preferably at least 90% by weight of the organic solvent is removed in step (ii) of the process. A preferred method for removing organic solvent in step (ii) is to add the polymer/solvent/LH-RH mixture from step (i) drop-wise into liquid nitrogen and remove the organic solvent from the frozen droplets by freeze-drying. Optionally further organic solvent may be removed from the resulting freeze dried material by, for example, vacuum drying or oven drying. Suitably the temperature during the vacuum or oven drying is from 20° C. to 65° C., for example from 25 to 60° C.

The polylactide-LH-RH analogue composition resulting from step (ii) of the process may be formed into a monolithic implant by, for example compression molding, injection molding or extrusion.

According to a further aspect of the present invention there is provided a method for treating a warm blooded animal (preferably a human) suffering from a condition treatable by an LH-RH analogue comprising administering thereto a monolithic implant according to the first aspect of the present invention.

According to another aspect of the present invention there is provided a monolithic implant according to the first aspect of the present invention for use as a medicament in the treatment of a condition treatable with an LH-RH analogue.

The monolithic implants according to the present invention are useful in the treatment of a wide variety of medical conditions requiring the administration of an LH-RH analogue. Such medical conditions include, but are not limited to, hormone dependent diseases such as prostate cancer, breast cancer, ovarian cancer, uterine cancer and testicular cancer, in gynaecology for the treatment of endometriosis, hysteroscopy and uterine fibroids, and for the treatment of fertility disorders.

The dose of LH-RH analogue required for the treatment of a particular condition will be dependent upon both the condition being treated and the animal to which it is administered. In the case of a human, a dose of LH-RH analogue suitable for release over a six month period ranges, for example from 10 to 40 mg, more preferably from 20 to 30 mg. The dose may be provided by means of a single monolithic implant or, particularly when a large dose of LH-RH analogue is required, by means of two or more monolithic implants.

According to a further aspect of the present invention, there is provided a method for administering an LH-RH analogue to a warm blooded animal, especially a human, comprising implanting (preferably subcutaneously) a monolithic implant according to the present invention in the warm blooded animal.

The monolithic implant may be implanted using conventional medical techniques. For example, subcutaneous implantation may be achieved using a needle or via a trochar or the like.

The invention is further illustrated by the following examples wherein all parts are by weight unless otherwise stated.

### BRIEF DESCRIPTION OF THE DRAWINGS

FIG. 1 shows the in vitro release of goserelin from monolithic implants comprising a single polylactide homopolymer with weight average molecular weight of 21 kDa (black squares as data points), “implant A” and 31 kDa (black diamonds as data points), “implant

B”; both implants have a 30% w/w loading of goserelin. Comparative curves are also shown showing the release from an implant consisting of a polylactide homopolymer (MW of 59 kDa) and 30% w/w goserelin loading (grey triangles as data points), “comparative A” and an implant consisting of a polylactide homopolymer (MW of 59 kDa) and 20% w/w goserelin loading (grey circles as data points), “comparative B”.

FIG. 2 shows the in vitro release of goserelin from monolithic implants comprising blends of polylactide homopolymers according to the present invention. Curve A (black diamonds as data points) corresponds to a blend of polylactides with a composition [75/25 wt % 21 kDa/31 kDa] having a mean MW of 23.5 kDa, “implant C”. Curve B (black squares as data points) corresponds to a blend of polylactides with the composition [75/25 wt % 12.5 kDa/59 kDa] having a mean MW of 24.1 kDa, “implant D”. In both cases the implants contained a loading of 30% w/w goserelin. Two comparative in vitro release profiles are also given. Curve C (grey triangles as data points) corresponds to a blend of polylactides with the composition [30/70 wt % 21 kDa/59 kDa] having a mean MW of 47.6 kDa and a drug loading of 30% w/w, “comparative C”. Curve D (grey circles as data points) corresponds to a blend of polylactides with the composition [75/25 wt % 12.5 kDa/31 kDa] having a mean MW of 17.1 kDa and a drug loading of 20% w/w, “comparative D”.

FIG. 3 shows a comparison of release profiles of goserelin from larger implants (approximately 3 times longer) corresponding to the formulations of implant B (31 kDa PLA, 30% goserelin) and implant C (blend of 75/25 wt % 21 kDa/31 kDa, 30% goserelin), with the release from implants B and C. The larger implants are labelled E and F, respectively.

FIG. 4 shows a simulated in vitro release profile corresponding to the predicted total release of goserelin from two implants, one prepared from 12.5 kDa polymer and the other comprised of 31 kDa polymer, both with a goserelin loading of 30% w/w.

### POLYLACTIDE POLYMERS

The Poly(dl lactide)s shown in Table 1 were prepared via ring opening of dl-lactide dimer in the presence of lactic acid and stannous octoate. A mixture of dl lactide and lactic acid in the appropriate quantities shown in Table 1 was added to a dry flask and purged with nitrogen. The mixture was then heated to 160° C. and stirred, and a small quantity of stannous octoate was added (150-300 μl for the polymers used to prepare the polymers herein). The temperature was then increased to 180° C. (or maintained at 160° C. in the case of polymer B) and stirring continued for a further hour. The mixture was maintained at this temperature, under stirring for a further 5 hours under a slow flow of nitrogen. The resulting polymer was taken up in glacial acetic acid and then re-precipitated into methanol. The resulting white solid was dried at 40° C. under a high vacuum and then post dried in the presence of potassium carbonate. The weight average molecular weight (MW) of the polymer was determined using size exclusion chromatography (SEC) using polymer solutions in Tetrahydrofuran (THF) with 2×30 cm² mixed bed ‘D’s PLGel columns (supplier Polymer Laboratories) which have a linear range of MW 200-400,000 Da. The PLGel packing material is a highly cross linked spherical polystyrene/divinylbenzene matrix. A Wyatt Optilab DSP refractometer was used for detection. The system is first calibrated using PL Easical PS-2 polystyrene calibrants with Mw in the range 580 to 400,000 Da.

TABLE 1

| | | | Stannous | |
|---|---|---|---|---|
| Polymer | dl-Lactide (g) | Lactic acid (μl) | octoate (μl) | MW (kDa) |
| Polymer A | 200 | 1600 | 300 | 21 |
| Polymer B | 100 | 400 | 300 | 31 |
| Polymer C | 200 | 2800 | 300 | 12.5 |
| Polymer D | 100 | 100 | 150 | 59 |

### EXAMPLE 1

### Implants Comprising a Single Polylactide Polymer

The implants shown in Table 2 were prepared by melt extrusion of freeze dried mixtures of drug and polymer. Batch sizes of up to 5 grams were prepared by dissolving the weight shown in Table 2 of goserelin acetate and the relevant polylactide from Table 1 in glacial acetic acid and drop freezing into liquid nitrogen. The resulting frozen spheroids were freeze dried for a minimum period of 24 hours using a freeze dryer (Edwards), to remove glacial acetic acid from the samples. The resulting solid was then put through a secondary drying step as follows. Temperature was ramped from 20° C. from 45° C. over an hour followed by dwell time of 5 hours. Temperature was then ramped again over 1 hour from 45° C. to 60° C. and then held at 60° C. for a period of 5 hours. Samples were retained in a vacuum dessicator prior to extrusion.

Drug/polymer blends prepared in this manner were extruded using a three piece, one gram capacity, stainless steel extruder consisting of a barrel/nozzle, base and die with a diameter of 2.3 mm. The extruder was heated using a pipe clamp, the temperature of which was controlled through a feedback circuit containing a thermocouple, located in thermal contact with the extruder barrel, and a custom built controller unit. Pressure was applied to the extruder die via a standard hydraulic KBr Press (Specac). Extrusion pressure was controlled manually. All extrudates were manufactured using the same nominal heating programme. The temperature of the extruder was initially raised to a nominal value of 95° C. over a period of 30-60 minutes, and then held at this temperature for a further 90-120 minutes. Extrusion of the drug/polymer melt was then performed by applying pressures of up to 1 Ton (nominal) to the extruder die. The resulting extrudates were cut into the required lengths. Dissolution tests were performed on two samples of each depot formulation, the weights of which are shown in Table 2. All release profiles shown represent the mean of 2 dissolution trials, run in parallel.

TABLE 2

| | | | Goserelin Acetate | |
|---|---|---|---|---|
| | Polymer | | Weight (g) & | |
| | from | Polymer | Nominal Peptide | Implant |
| Implant | Table 1 | Weight (g) | Loading (% w/w) | Weight (mg) |
| Implant A | Polymer A | 1.40 | 0.66, | 11.33, 8.53 |
| | | | 30% loading | |
| Implant B | Polymer B | 1.40 | 0.66, | 11.00, 12.81 |
| | | | 30% loading | |
| Comparative A | Polymer D | 1.40 | 0.66, | 13.61, 9.17 |
| | | | 30% loading | |
| Comparative B | Polymer D | 2.40 | 0.70, | 17.34, 18.77 |
| | | | 20% loading | |
| Implant E | Polymer B | 3.50 | 1.69, | 37.71, 37.73 |
| | | | 30% loading | |

Release Profile

The rate of release of the goserelin in vitro was measure by placing the implants shown in Table 2 in 10 ml (or 50 ml for larger implants E and F) of phosphate buffered saline (PBS)

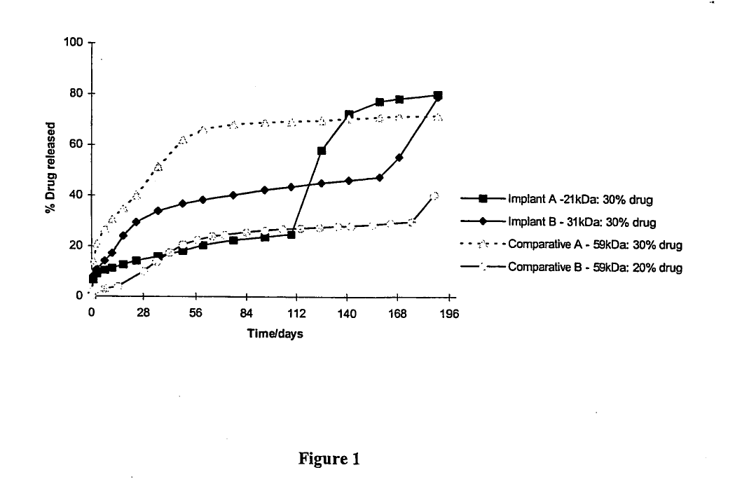

comprising 1.38 g Na₂HPO₄, 0.19 g KH₂PO₄, 8.00 g NaCl and 0.20 g NaN₃ per litre of deionised water (adjusted to pH 7.4+/−0.05 using 0.1M HCl) and then stored in an incubator at a temperature of 37° C. At regular time intervals, 4 ml (or 20 ml for larger implants E and F) samples of dissolution medium were withdrawn from each sample vial. An equal volume of fresh PBS was returned to each vial after sampling. Sampling was either performed manually, with a pippetor, or by means of a robot (Gilson 215 Liquid Handler). The goserelin content of all dissolution media samples were measured using an isocratic HPLC method (Vydac C18 column, mobile phase 80/20 v/v mixture of (˜80/20/0.1% v/v) Water/Acetonitrile/Trifluoracetic acid) and (˜40/60/0.1% v/v) Water/Acetonitrile/Trifluoracetic acid). Goserelin concentrations were calculated on the basis of peak area, against external calibration standards, prepared by dissolving weighed quantities of goserelin in pure water. The release profiles, calculated from the measured goserelin concentrations are shown in FIG. 1.

FIG. 1 shows that Implants A and B released the goserelin continuously over a period of more than six months (180 days). However, Comparative B (high molecular weight polymer and low LH-RH loading) exhibited a long induction period over the first 14 days when little or no goserelin was released. In addition, this depot only releases around 30% w/w of its payload up to around 170 days. Comparative B (high molecular weight polylactide) shows an initial rapid release of drug followed by a sustained plateau in which little or no drug was released.

### EXAMPLE 2

### Implants Comprising a Blend of Polylactide Polymers

The implants shown in Table 3 were prepared in an analogous way to those described in Example 1 except that the blend of polymers shown in Table 3 was used.

TABLE 3

| | | | | Goserelin Acetate | |
|---|---|---|---|---|---|
| | | Polymer | Mean molecular | Weight (g) & | Implant |
| | Polymer | Weight | weight | Nominal Peptide | Weight |
| Implant | from Table 1 | (g) | (kDa) | Loading (% w/w) | (mg) |
| Implant C | Polymer A | 1.05 | 23.5 | 0.66, | 11.31, 8.65 |
| | Polymer B | 0.35 | | 30% loading | |
| Implant D | Polymer C | 1.05 | 24.1 | 0.66, | 10.22, 11.00 |
| | Polymer D | 0.35 | | 30% loading | |
| Comparative C | Polymer A | 0.30 | 47.6 | 0.46, | 12.16, 15.93 |
| | Polymer D | 0.69 | | 30% loading | |
| Comparative D | Polymer B | 0.40 | 17.1 | 0.44, | 11.40, 11.28 |
| | Polymer C | 1.20 | | 20% loading | |
| Implant F | Polymer A | 2.63 | 23.5 | 1.69, | 34.96, 37.16 |
| | Polymer B | 0.88 | | 30% loading | |

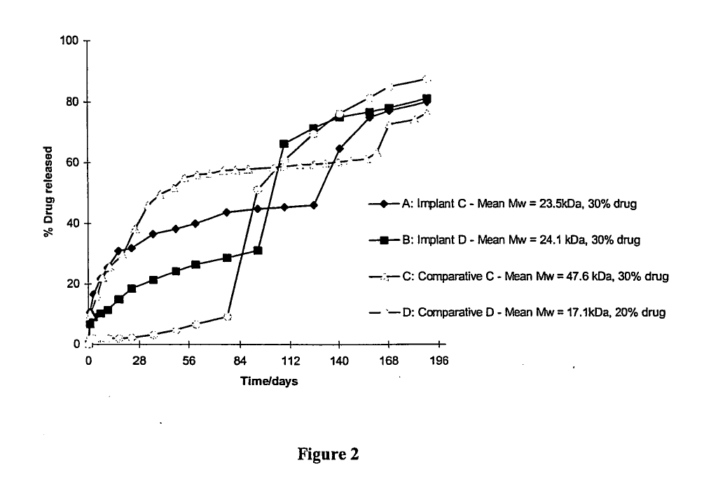

The release profile of the goserelin free base from each implant was measured in-vitro as described in Example 1. The release profiles are shown in FIG. 2. FIG. 2 clearly shows than Implants C and D (according to the present invention) released the goserelin continuously for a period of at least 6 months. On the other hand the Comparative Implants C and D both exhibited long periods during which there was little or no release of drug.

### EXAMPLE 3

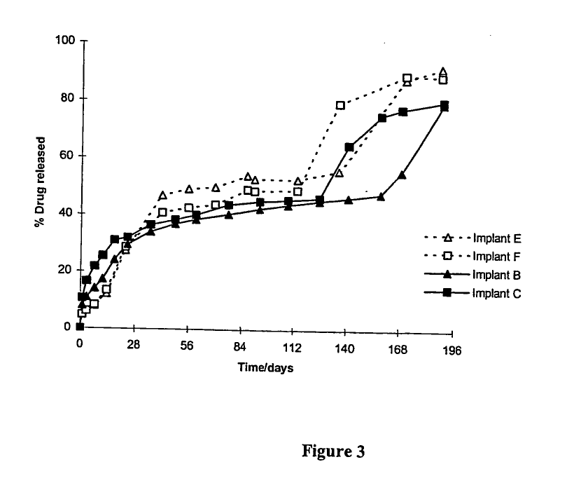

The effect of depot size upon release profile was explored by measuring the in vitro release of goserelin from selected depot formulations, using implants approximately 3 times larger than those used in Examples 1 and 2 above. FIG. 3 shows two comparative plots;
i. Release profile of Implant B (small implant) versus Implant E (large implant) 31 kDa PLA, 30% goserelin
ii. Release profile of Implant C (small implant) versus Implant F (large implant) 75/25 wt % 21 kDa/31 kDa PLA, 30% goserelin
   It can be seen that for these particular formulations, the larger depots have a similar release profiles to those of the smaller implant samples, suggesting that depot size does not significantly affect the percentage release of drug as a function of time.

### EXAMPLE 4

The release of goserelin resulting from the use of two different half sized implants was simulated for a combination of 12.5 kDa and 31 kDa implants, both with a 30% w/w goserelin payload. This was done by summing the measured percentage release from a 12.5 kDa depot and a 31 kDa depot at each time point and dividing this value by two to give the predicted release from two half depots.

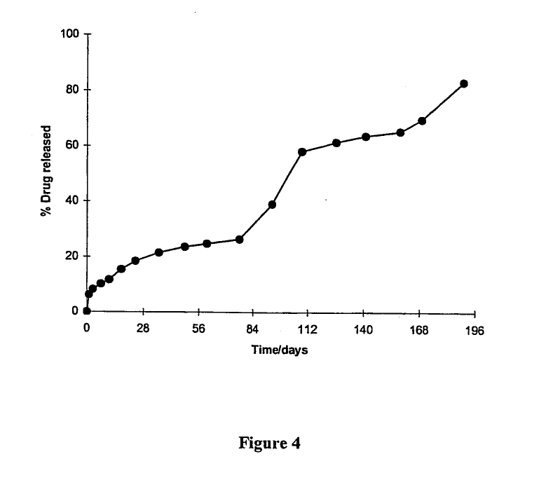

FIG. 4 shows the simulated in-vitro release profile corresponding to the predicted total release of goserelin from two implants, one prepared from 12.5 kDa polymer and the other comprised of 31 kDa polymer, both with a goserelin loading of 30% w/w. A near zero-order release profile is obtained.

1. A monolithic implant comprising:
(i) a polylactide polymer having a weight average molecular weight of from 12,000 Daltons to 40,000 Daltons; and
(ii) from 25 to 40% by weight, based upon the total weight of the implant, of an LH-RH analogue;
wherein the monolithic implant continuously releases the LH-RH analogue over a period of at least six months when placed in an aqueous physiological-type environment.

2. A monolithic implant according to claim 1, wherein the LH-RH analogue is selected from leuprorelin, buserelin, triptorelin, and goserelin.

3. A monolithic implant according to claim 1, wherein the polylactide polymer is a homopolymer, wherein all the repeat units of the polymer are of Formula (1)

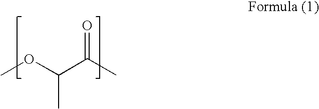

4. A monolithic implant according to claim 3, wherein the repeat units of Formula (1) comprise a mixture of repeat units in the L-and-D-configurations, and the ratio of L- to D-units is from 25:75 to 75:25.

5. A medicament comprising two monolithic implants of any one of the preceeding claims, wherein the two implants release the LH-RH analogue at different rates when placed in an aqueous physiological environment.

6. A process for the preparation of an implant according to claim 1, comprising:
(i) preparing a solution or dispersion of the LH-RH analogue in a solution comprising a polylactide polymer and a suitable organic solvent;
(ii) removing substantially all of the organic solvent from the solution or dispersion formed in step (i); and
(iii) forming the product of step (ii) into a monolithic implant of the required dimensions.

7. A method for treating a warm blooded animal suffering from a condition treatable by an LH-RH analogue, comprising administering thereto a monolithic implant according to claim 1.

8. A method for administering an LH-RH analogue to a warm blooded animal, comprising implanting a monolithic implant according to claim 1 in the warm blooded animal.

9. A method for administering an LH-RH analogue to a warm blooded animal, comprising subcutaneously implanting a monolithic implant according to claim 1 in the warm-blooded animal.
